(19) United States
(12) Patent Application Publication
(10) Pub. No.: US 2021/0088517 A1
(43) Pub. Date: Mar. 25, 2021

(54) MULTIPLEX HIGH-THROUGHPUT FLOW CYTOMETRY DETECTION OF SARS-COV-2-SPECIFIC IgG, IgA AND IgM

(71) Applicant: RayBiotech Life, Peachtree Comers, GA (US)

(72) Inventors: Ruo-Pan Huang, Johns Creek, GA (US); Benyue Zhang, Lilburn, GA (US)

(21) Appl. No.: 17/036,042

(22) Filed: Sep. 29, 2020

(51) Int. Cl. G01N 33/569

(52) U.S. Cl. CPC G01N 33/56983; G01N 2469/20; G01N 2333/165

(57) ABSTRACT

A multiplex bead-based high-throughput high sensitivity diagnosis method is provided that can detect human IgG, IgA and IgM directed against SARS-CoV-2 simultaneously, with minimum false positive results is provided. Instead of comparing the absolute read signal, this kit introduces an internal control as background reference for each specific sample. By comparing the ratio of signals between viral antigen-coated beads and control protein-coated beads the real signal due to anti-viral Ig can be determined.

### SEQUENCE LISTING

This application contains a sequence listing filed in electronic form as an ASCII.txt file entitled “2208031090_ST25” created on Sep. 21, 2020. The content of the sequence listing is incorporated herein in its entirety.

### TECHNICAL FIELD

The present disclosure is generally related to methods of multiplex cytometry for the detection of SARS-CoV-2-specific IgG, IgA and IgM. The present disclosure is also generally related to kits for the performance of the methods of the disclosure.

### BACKGROUND

The world is challenged by the pandemic caused by a new coronavirus, SARS-CoV-2. SARS-CoV-2 infects humans and can cause serious pneumonia complicated by extensive inflammatory cytokines production (a “cytokine storm”). The virus-associated disease has typical symptoms of pneumonia accompanied by several other symptoms e.g. muscle pain, headache and sore throat and is most commonly referred to as COVID-19.

Currently there are no known effective therapeutic treatments for a COVID-19 infection. Therefore, the prognosis largely depends on the efficacy of the host's immune system. Early diagnostic kits directed against virus-specific IgM and IgG antibodies, or q-RT-PCR kits detecting viral levels have been developed, but the supply has been in a great shortage in the United States. In addition, the high percentage of false positive results, mainly caused by high levels of immunoglobulin in certain patients, has also been a challenge for serum anti-virus immunoglobulin (Ig) detection.

### SUMMARY

Embodiments of a multiplex bead-based high-throughput high sensitivity diagnosis method that can detect human IgG, IgA and IgM directed against SARS-CoV-2 simultaneously, with minimum false positive results is provided. Instead of comparing the absolute read signal, this kit introduces an internal control as background reference for each specific sample. By comparing the ratio of signals between viral antigen-coated beads and control protein-coated beads the real signal due to anti-viral Ig can be determined.

One aspect of the disclosure encompasses embodiments of a method of detecting an immune response to SARS-CoV-2, the method comprising: (a) incubating a biological sample from a subject suspected of having been exposed to SARS-CoV-2 virus with a multiplex bead array, wherein the multiplex bead array comprises a plurality of bead populations wherein each bead population is characterized as small, medium, or large based on the size of the beads and grouped according to the intensity of emission of fluorescence therefrom, and wherein the multiplex beads are coated with a protein of SARS-CoV-2, or a fragment thereof, and the sample is incubated individually with a single bead population of the multiplex array of beads; (b) combining and washing the incubated bead populations; (c) adding a mixture of anti-human immunoglobulin (Ig) antibodies to the combined bead populations from step (b), wherein the mixture of anti-human Ig antibodies can comprise at least one anti-human IgG antibody, at least one IgA antibody, and at least one IgM antibody, and wherein the anti-human IgG, IgA, and IgM antibody or antibodies are differentially labeled to distinguish the anti-human IgG antibody or antibodies, the anti-human IgA antibody or antibodies, and the anti-human IgM antibody or antibodies from each other, and wherein the anti-human IgG antibody or antibodies, the anti-human IgA antibody or antibodies, and the anti-human IgM antibody or antibodies can be conjugated to different fluorochromes detectable by, and distinguishable from each other, by flow cytometry, and then washing the incubated bead population; (d) repeating steps (a)-(c), wherein the multiplex beads can be coated with a bovine serum albumin, or a fragment thereof; (e) individually analyzing the combined bead populations from steps (c) or (d) with respect to the bead size and the intensity of the emission of a fluorescent signal from each of the different fluorochromes and calculating the ratio of the mean fluorescence intensity (MFI) derived from the results from the combined bead populations coated with a protein of SARS-CoV-2, or a fragment thereof, of step (c) and from the results from the combined bead populations coated with bovine serum albumin of step (d); and (f) determining the relative levels of human IgG, IgA, and IgM bound to the SARS-CoV-2 protein, or a fragment thereof, thereby determining the immune response of the subject to a SARS-CoV-2 infection.

In some embodiments of this aspect of the disclosure, the beads can be red fluorescent beads.

In some embodiments of this aspect of the disclosure, each bead population can be in an individual volume in a reaction vessel.

In some embodiments of this aspect of the disclosure, the reaction vessel containing an individual bead population of the multiplex array can be a well of a multiwell-plate.

In some embodiments of this aspect of the disclosure, the method can be a high-throughput assay, wherein each bead population is dispensed into the wells of a single multiwell plate and wherein each well having a bead population coated with a protein of SARS-CoV-2, or a fragment thereof can be paired with a well having a bead population coated with bovine serum albumin.

In some embodiments of this aspect of the disclosure, the protein of SARS-CoV-2 is a SARS-CoV-2 spike protein, a nucleocapsid protein, or a fragment thereof, or any combination thereof.

In some embodiments of this aspect of the disclosure, the protein of SARS-CoV-2 is a SARS-CoV-2 spike protein, or a fragment thereof.

In some embodiments of this aspect of the disclosure, the SARS-CoV-2 spike protein is from amino acid positions Arg319 to Phe541 of the receptor-binding domain (RBD)

In some embodiments of this aspect of the disclosure, the labeling moieties can be selected from the group consisting of fluorescein isothiocyanate (FITC, green), Cy2, Cy3, Cy3.5, Cy5, Cy5.5 Cy7, Cy7.5, Texas Red, an Alexa Fluor, a HILYTE™ Fluor, a DYLITE™ Fluor, RayBright® V450, RayBright® B488, and Red Fluorescent Protein (R-PE; R-Phycoerythrin).

In some embodiments of this aspect of the disclosure, the anti-human IgG antibody or antibodies are conjugated to RayBright® V450, the anti-human IgA antibody or antibodies are conjugated to RayBright® B488, and the anti-human IgM antibody or antibodies are conjugated to R-Phycoerythrin.

Another aspect of the disclosure encompasses embodiments of a kit comprising vessels containing a series of size and fluorescent-intensity sorted beads, wherein the beads are coated with at least one polypeptide or fragment thereof derived from SARS-CoV-2, and vessels containing an anti-human IgG antibody, an anti-human IgA antibody, and an anti-human IgM antibody, or antigen-binding fragments thereof, and instructions for the use of the reagents of the kit in a method for the multiplex high-throughput flow cytometry detection of SARS-CoV-2-specific IgG, IgA and IgM antibodies.

In some embodiments of this aspect of the disclosure, the polypeptide or fragment thereof derived from SARS-CoV-2 is at least one of a SARS-CoV-2 spike protein, or a fragment thereof, and a SARS-CoV-2 nucleocapsid protein or a fragment thereof.

In some embodiments of this aspect of the disclosure, the protein of SARS-CoV-2 is a SARS-CoV-2 spike protein.

In some embodiments of this aspect of the disclosure, the SARS-CoV-2 spike protein is from amino acid positions Arg319 to Phe541 of the receptor-binding domain (RBD)

In some embodiments of this aspect of the disclosure, the kit comprises multiplex beads conjugated with SARS-CoV-2 spike protein (R1-S to R25-S), multiplex beads conjugated with bovine serum albumin (R1-CTL to R25-CTL), assay diluent, wash buffer, a V-shaped 96-well microplate, RayBright.RTM V450 labeled goat-anti-human IgG (Fc), RayBright® B488 goat-anti-human IgA (Fc), and R-PE goat-anti-human IgM (Fc).

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates the assay of the high-throughput flow cytometry measurement of human SARS-CoV-2-specific IgG, IgA and IgM of the disclosure. The assay is a two-step Multiplex Bead Antigen Array with internal control beads.

Beads are conjugated with either SARS-CoV-2 S1 antigen (S1 antigen beads) or bovine serum albumin (BSA) (control beads) and incubated with samples containing IgM, IgG or IgA antibodies against SARS-CoV-2 virus, followed by staining with fluorochrome-conjugated goat anti-human IgM, IgG and IgA secondary antibodies. Anti-viral immunoglobulin levels are analyzed on a multi-color flow cytometer.

FIG. 2 illustrates the arrangement of samples and multiplex beads in a series of 96-well plates according to the method of the disclosure.

FIG. 3 illustrates the size distribution of multiplex beads used in the methods of the disclosure. Bead ID R1-R7: Large Size Beads; Bead ID R8-R17: Medium Size Beads; Bead ID R18-R25: Small Size Beads.

FIG. 4 illustrates multiplex beads by size and color intensity distribution. Each bead of R1-R25 can be conjugated with SARS-CoV-2 Spike protein (antigen beads) or BSA as control protein (control beads), 50 bead populations.

FIG. 5 illustrates a representative image of flow cytometer set-up for a test using large, and medium beads.

FIG. 6 illustrates a representative image of flow cytometer set-up for a test using large, medium and small red beads.

FIGS. 7A and 7B illustrate a sample graph of testing of 13 SARS-CoV-2 patient serum samples with multiplex beads according to the disclosure.

After incubation, 13 antigen beads or 13 control beads were pooled together and incubated with RayBright® V450-labeled anti-human IgG, RayBright® B488-labeled anti-human IgA and R-PE-labeled anti-human IgM. Mixed beads were analyzed by a FACS Celesta on Phycoerythrin (R-PE) and allophycocyanin (APC) channels.

FIG. 7A illustrates when 13 SARS-CoV-2 patient sera are incubated with 13 Sars-Cov-2 S1 antigen beads, respectively.

FIG. 7B illustrates when 13 SARS-CoV-2 patient sera were incubated with 13 control beads.

FIG. 8 illustrates a table showing representative positive samples analyzed in the same plate as 4 replicates. For each of IgM, IgG and IgA, intra-plate coefficient of variation (CV) for mean fluorescence intensity (MFI) readings, and the MFI ratio between antigen-beads and control beads are shown.

FIG. 9 illustrates a table showing representative positive samples analyzed in 4 different plate as 4 replicates. For each of IgM, IgG and IgA, inter-plate coefficient of variation (CV) for mean fluorescence intensity (MFI) readings, and the MFI ratio between antigen-beads and control beads are shown.

FIG. 10 illustrates the amino acid sequence of the surface spike glycoprotein of SARS-CoV-2 (SEQ ID NO: 1), amino acids R319-F541 of the receptor binding domain (RBD) (SEQ ID NO: 2), and amino acids M697-P1213 (SEQ ID NO: 3) of the S2 region of SEQ ID NO: 1.

FIG. 11 illustrates the amino acid sequence M1-A419 of the SARS-CoV-2 (SEQ ID NO: 4), of the SARS-CoV-2-derived nucleocapsid protein.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Abbreviations

Bovine Serum Albumin, BSA; Fluorescence-activated Cell Sorting, FACS; Phycoerythrin, R-PE; Allophycocyanin, APC; coefficient of variation, CV; mean variation of fluorescence intensity, MFI;

### Definitions

The term “fluorochrome” as used herein refers to photoreactive chemicals that can absorb energy via the interaction of an orbital electron in the molecule's atomic structure with a photon of light. In flow cytometry, light energy is typically derived from a monochromatic laser, which is used to produce the predictable excitation of an irradiated fluorochrome, followed by the subsequent emission of a photon as the excited electron relaxes back to its original ground state.

The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far red part of the spectrum; quantum yield is 0.28. FW=792. Suitable fluorophores(chromes) for the probes of the disclosure may be selected from, but not intended to be limited to, fluorescein isothiocyanate (FITC, green), cyanine dyes Cy2, Cy3, Cy3.5, Cy5, Cy5.5 Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.), HILYTE™ Fluors (AnaSpec), and DYLITE™ Fluors (Pierce, Inc). Advantageously, the fluorescent dyes of the methods of the disclosure may be, but are not limited to, RayBright® V450 RayBright® B488, and Red Fluorescent Protein (R-PE; R-Phycoerythrin) (Raybiotech Life Inc., Peachtree Corners, Peachtree Corners, U.S.A).

The term “specific binding” as used herein refers to the specific recognition of one molecule, of two different molecules, compared to substantially less recognition of other molecules. Generally, the molecules have areas on their surfaces or in cavities giving rise to specific recognition between the two molecules. Exemplary of specific binding are antibody-antigen interactions.

The term “antibody” as used herein refers to an immunoglobulin which specifically binds to and is thereby defined as complementary with a particular spatial and polar organization of another molecule. The antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, IgY, etc. Fragments thereof may include Fab, Fv and F(ab′)2, Fab′, scFv, and the like. In addition, aggregates, polymers, and conjugates of immunoglobulins or their fragments can be used where appropriate so long as binding affinity for a particular molecule is maintained.

Antibodies may be derived from any source, including, but not limited to, murine spp., rat, rabbit, chicken, human, or any other origin (including humanized antibodies). Techniques for the generation of antibodies that can specifically recognize and bind to are known in the art. The term “antigen” as used herein refers to any entity that binds to an antibody and induces at least one shared conformational epitope on the antibody. Antigens can be proteins, peptides, antibodies, small molecules, lipid, carbohydrates, nucleic acid, and allergens. An antigen may be in its pure form or in a sample in which the antigen is mixed with other components. In particular, the methods of the present disclosure are intended to detect human or animal immunoglobulins that specifically recognize and bind to epitopes of the S and/or N polypeptides of the SARS-CoV-2 virus.

The term “flow cytometry (FCM) as used herein refers to a technique used to detect and measure physical and chemical characteristics of a population of cells or particles.

In this process, a sample containing cells or, as in the present disclosure, beads suspended in a fluid and injected into the flow cytometer instrument. The sample is focused to ideally flow one cell at a time through a laser beam, where the light scattered is characteristic to the beads and their components. The multiplex beads of the present disclosure are often labeled with fluorescent markers so light is absorbed and then emitted in a band of wavelengths. A flow cytometry analyzer is an instrument that provides quantifiable data from a sample. Other instruments using flow cytometry include cell sorters which physically separate and thereby purify cells of interest based on their optical properties.

The term “cytometric bead array” as used herein refers to a method similar to ELISA sandwich assays, wherein cytometric bead array (CBA) assays use multiple bead populations typically differentiated by size and different levels of fluorescence intensity to distinguish multiple analytes in a single assay. The amount of the analyte captured (in the methods of the present disclosure that is antibodies in a sample from an animal or human subject specific for a SARS-CoV-2-derived antigen) can be detected via a labeled antibody specifically binding to the animal or human immunoglobulin (IgA, IgG, and/or IgM bound to the SARS-CoV-2-derived antigen on the beads). Concentrations of a protein of interest in the samples can be obtained by comparing the fluorescent signals to those of a standard curve generated from a serial dilution of a known concentration of the analyte.

The term “Severe Acute Respiratory Syndrome Coronavirus 2 (SARS-CoV-2)” as used herein refers to is the strain of coronavirus that causes coronavirus disease 2019 (COVID-19), the respiratory illness responsible for the COVID-19 pandemic. Colloquially known as simply the coronavirus, it was previously referred to by its provisional name, 2019 novel coronavirus (2019-nCoV), and has also been called human coronavirus 2019 (HCoV-19 or hCoV-19) . SARS-CoV-2 is a Baltimore class IV positive-sense single-stranded RNA virus that is contagious in humans. It is the successor to SARS-CoV-1, the strain that caused the 2002-2004 SARS outbreak.

Each SARS-CoV-2 virion is 50-200 nm in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. The spike protein, which has been imaged at the atomic level is responsible for allowing the virus to attach to and fuse with the membrane of a host cell; specifically, its 51 subunit catalyzes attachment, the S2 subunit fusion.

SARS-CoV-2 has sufficient affinity to the receptor angiotensin converting enzyme 2 (ACE2) on human cells to use them as a mechanism of cell entry. Studies have shown that SARS-CoV-2 has a higher affinity to human ACE2 than the original SARS virus strain.

Initial spike protein priming by transmembrane protease, serine 2 (TMPRSS2) is essential for entry of SARS-CoV-2. After a SARS-CoV-2 virion attaches to a target cell, the cell's protease TMPRSS2 cuts open the spike protein of the virus, exposing a fusion peptide in the S2 subunit, and the host receptor ACE2. After fusion, an endosome forms around the virion, separating it from the rest of the host cell. The virion escapes when the pH of the endosome drops or when cathepsin, a host cysteine protease, cleaves it. The virion then releases RNA into the cell and forces the cell to produce and disseminate copies of the virus, which infect more cells.

### Discussion

The methods of the disclosure encompass the use of a multiplex micro-bead technology that uses small volumes of patient sample and reagents, is compatible to microtiter tray analysis platforms for high throughput, and can be as rapid an assay as possible without the need for additional technical supervision. Reagents may be added to a single vessel such as, for example, a microtiter plate, incubated, and analyzed by a flow cytometer.

The size characteristics and/or a combination of size and fluorescent properties of the beads distinguish them from each other. On flow cytometers, this is accomplished by utilizing Forward Scatter properties (size) together with Side Scatter (SSC), which is a measurement of the refractive properties of particles passing in front of a light source, which is generally a laser.

These signals are combined to form scatter plots by the host computer, which may be utilized in the identification of bead population(s). Furthermore, inherent or secondary bead fluorescence may be used as an additional means of “gating” selected populations. In combination with each other, multiple properties of size, side scatter, and fluorescence, create unlimited possibilities of analytes detected per sample.

Specific volumes of bead suspensions, pre-diluted patient serum, and fluorophore-secondary antibody conjugate can be simultaneously mixed together in a single reaction vessel, incubated and analyzed in a very short period of time. Proportional bead coating and antigen/bead ratios have been determined allowing the beads to be distinct populations when viewed on the flow cytometers. Similarly, the “normal” or negative controls used with these assay systems are clearly distinguished from “positive” samples. Results are reported either as a qualitative result (positive or negative), using specific mean channel cut-off or as semi-quantitative values by dividing the mean channel fluorescence of the positive sample by the mean channel of the negative control. This creates the potential for monitoring serum titers of the specific analyte. Quantitative results may also be incorporated by utilizing known multiple positive control standards, which may form concentration curves when plotted on a graph of result versus concentration value.

One aspect of the disclosure encompasses embodiments of antibody detection kits that simplify the process of analyzing samples for the presence of SARS-CoV-2-specific antibodies in the serum of a subject. Beads can be coated with the S or N polypeptides, or fragments thereof to their exterior. Advantageously, there can be multiple sized beads. Each bead may be coated with one or more SARS-CoV-2-specific antigens and the beads are impregnated with specific dyes.

The beads can be aliquoted into reaction vessels and have specific amounts of pre-diluted sample added to them. Shortly thereafter, an indicator conjugate comprising a fluorophore conjugated to an anti-immunoglobulin antibody is added to the mixture, incubated and analyzed on the flow cytometer. Bead/sample mixtures are aspirated by the flow cytometer, pass through a flow cell, and are analyzed and distinguished by the bead(s)scatter properties when presented in front of a light source produced from laser(s) found within the flow cytometer itself. Bead populations are distinguished from each other by their size, light scattering, and fluorescent properties. Each bead has a unique scatter characteristic, which is identified by forward angle light scatter (size) together with side angle scatter (refractal) properties. These signals are converted into a digital signal, which are then graphically plotted on a two-dimensional histogram where each population may be delineated by drawing specific “gate(s)”, or windows. Information (for example, fluorescence) regarding events within these “gate(s)” is then transmitted to other individual plots, or histograms, to determine their properties (for example, positive or negative, bright or dim, etc.). Negative control samples are initially analyzed to determine inherent fluorescent properties, or background. This signal, or mean channel fluorescence, will be the denominator of the assay result itself. Positive samples form reaction complexes with the beads and “shift” the position (mean channel fluorescence) of the population in relation to that of the negative control on the histogram.

Mean channel fluorescent measurements are defined as the relative position of the population of the beads found on a histogram. The scaling of these histograms may be different depending on the model of flow cytometer used. However, most flow cytometers have the ability to scale linear histograms from 0 to 1023 channels. This number may be utilized as an indicator of the relative degree of “positivity” to that of a normal or “negative” sample, therefore allowing the potential for semi-quantitative results.

Micro-bead technologies are analogous to microarrays except that the features are spatially segregated on different beads or particles. The analysis can be formatted like a microarray, for example, with the beads arrayed or embedded on the surface or in wells of a device such as a microscope slide or plate. The analysis can alternatively be performed with the beads suspended in a solution, for example. The working density of features for micro-bead technologies is potentially far greater than for microarrays, depending primarily on the minimum usable bead size and maximum usable bead concentration or density.

The beads of the methods of the disclosure can made of the same material such as poly(methyl methylacrylate) (PMMA) polystyrene, or latex. However, other polymeric materials are acceptable including polymers selected from the chemical group consisting of carbohydrate-based polymers, polyaliphatic alcohols, poly(vinyl) polymers, polyacrylic acids, polyorganic acids, polyamino acids, co-polymers, block co-polymers, tertpolymers, polyethers, naturally occurring polymers, polyimide, surfactants, polyesters, branched polymers, cyclo-polymers, polyaldehydes and mixtures thereof More specifically, brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, or combinations thereof are preferable. Representative combination polymers of which the polymeric particles are composed include for example poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio). Most of beads formed from synthetic polymers such as polystyrene, polyacrylamide, polyacrylate, or latex are commercially available from numerous sources such as Bio-Rad Laboratories (Richmond, Calif.) and LKB Produkter (Stockholm, Sweden). Beads formed from natural macromolecules and particles such as agarose, crosslinked agarose, globulin, deoxyribose nucleic acid, and liposomes are commercially available from sources such as Bio-Rad Laboratories, Pharmacia (Piscataway, N.J.), and IBF (France). Beads formed from copolymers of polyacrylamide and agarose are commercially available from sources such as IBF and Pharmacia. Particularly advantageous for use in the methods of the present disclosure are fluorescent beads coated with Covid-19-specific antigens, in particular the S and/or N polypeptides or fragments thereof such as from Raybiotech Inc., Peachtree Corners, Ga., U.S.A)

In general, the present disclosure provides the production of a plurality of substrates (e.g., beads) with different polypeptide or peptide targets attached thereto. The different polypeptides or peptides attached to the beads are typically produced off-line and can then be bound to beads in separate reactors, in a mechanical process of mixing solutions containing to form the dockerin-cohesin binding pair. This can be done in separate test tubes, vials or wells of a microtiter plate for example.

For the purposes of the methods of the disclosure, it is most advantageous for the SARS-CoV-2-derived antigen to be derived from the Spike protein of the virus. The full-length expressed protein has the amino acid sequence SEQ ID NO: 1 (Accession No: QHD43416) with amino acids V16-Q690 being the sequence minus a leader sequence. A most advantageous polypeptide derived from the spike protein is a fragment encompassing the Receptor Binding Domain (RBD) such as, but not limited to the amino acids R319-F541 (SEQ ID NO: 2) or antigenic fragments thereof that can have affinity with, and bound by, an anti- SARS-CoV-2-specific antibody. Determination of antigenic fragments of SEQ ID NO: 2 that can be useful in the methods of the disclosure can be obtained and confirmed to bind anti-SARS-CoV-2 antibodies by methods well-known to those of skill in the arts.

The methods of the disclosure may further be adapted by the use of the spike protein region 2 (amino acids M697-P1213 (SEQ ID NO: 3), or antigenic fragments thereof, or amino acids M1-A419 of the SARS-CoV-2-derived nucleocapsid protein (SEQ ID NO: 4 (Accession No: QHD43423)), or antigenic fragments thereof.

The present disclosure further provides embodiments of kits for practicing the screening methods of the disclosure. By “kit” is intended any manufacture (e.g., a package or a container) comprising at least one reagent for specifically detecting the presence of a SARS-CoV-2-specific antibody in a sample from a human or animal subject. The kit may be promoted, distributed, or sold as a unit for performing the methods of the present invention. Additionally, the kits may contain a package insert describing the kit and methods for its use.

In some embodiments, kits are for use in screening for identifying patients with at least one anti-SARS-CoV-2 antibody. Chemicals for the detection of anti-SARS-CoV-2 antibody binding to the bead-bound SARS-CoV-2 antigen by multiplex bead-based immunoassay format may be further included in a kit of the disclosure.

One of skill in the art will further appreciate that any or all steps in the screening methods of the invention could be implemented by personnel or, alternatively, performed in an automated fashion. That is, the methods can be performed in an automated, semi-automated, or manual fashion. Furthermore, the methods disclosed herein can also be combined with other methods known or later developed to permit a more accurate identification of patients having a SARS-CoV-2 infection or having been exposed to the SARS-CoV-2 virus.

The disclosure provides embodiments of a multiplex bead-based high-throughput high-sensitivity method, and kits to enable the method, to simultaneously detect human IgG, IgA and IgM antibodies directed against SARS-CoV-2 and which result in surprisingly few false positive results compared to existing methods. Instead of comparing the absolute read signal, the method introduces an internal control as a background reference for each specific sample. By comparing the ratio of signals between viral antigen-coated beads and control protein-coated beads, the real signal caused by anti-viral immunoglobulins can be determined.

The present disclosure encompasses embodiments of a method and kit that allows for the detection of human anti-SARS-CoV-2 IgG, IgA and IgM antibodies and which can be performed by a flow cytometer with Violet laser (V450), blue laser (FITC and PE channel) and red laser (APC channel). The minimum sample volume can be as little as 1mL that is diluted to as much as 1:8000 and provided results in as little as 2 hours.

Embodiments of the kit can allow for as many as 1200 human serum samples to be tested for anti-SARS-CoV-2 IgG, IgA and IgM in about 2 hours, a significant advance on the currently available 2 hours anti-SARS-CoV-2 antibody detection systems.

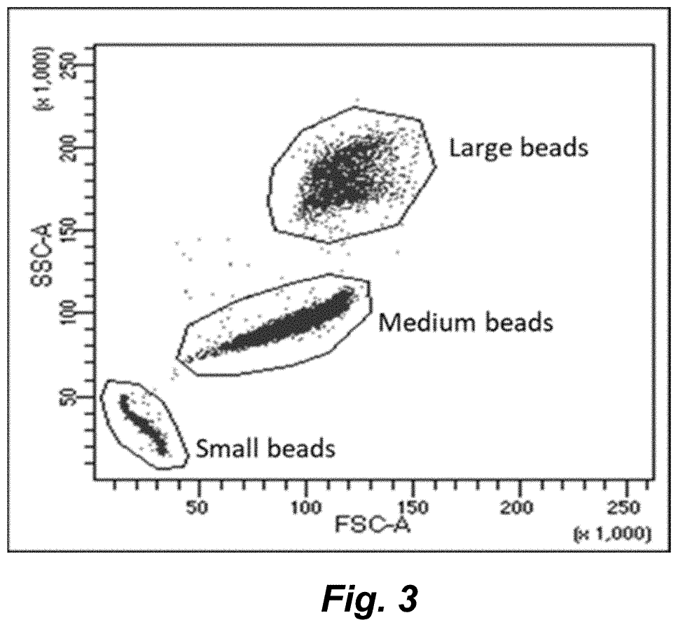

The assay of the disclosure can advantageously use, but are not limited to, suitable multiplex beads obtained from Raybiotech Life Inc, Peachtree Corners, Ga., U.S.A. and can be comprised of three size groups, large, medium and small. Target-specific beads are grouped as shown in FIG. 3 as Bead ID R1-R7: large; Bead ID R8-R17: medium; and Bead ID R18-R25: small.

The R1-R25 beads used in an embodiment of the method of the disclosure are conjugated either with an isolated SARS-CoV-2 spike protein, or fragment, thereof (Antigen-beads) or bovine serum albumin (BSA) (Control beads). Each kit of the disclosure, therefore, is provided with multiples of 25 sets of beads conjugated with either viral spike protein or BSA (Table 1).

TABLE 1

| Bead ID | Viral protein | Bead ID | Control protein |
|---|---|---|---|
| R1-S | Spike | R1-CTL | BSA |
| R2-S | Spike | R2-CTL | BSA |
| R3-S | Spike | R3-CTL | BSA |
| R4-S | Spike | R4-CTL | BSA |
| R5-S | Spike | R5-CTL | BSA |
| R6-S | Spike | R6-CTL | BSA |
| R7-S | Spike | R7-CTL | BSA |
| R8-S | Spike | R8-CTL | BSA |
| R9-S | Spike | R9-CTL | BSA |
| R10-S | Spike | R10-CTL | BSA |
| R11-S | Spike | R11-CTL | BSA |
| R12-S | Spike | R12-CTL | BSA |
| R13-S | Spike | R13-CTL | BSA |
| R14-S | Spike | R14-CTL | BSA |
| R15-S | Spike | R15-CTL | BSA |
| R16-S | Spike | R16-CTL | BSA |
| R17-S | Spike | R17-CTL | BSA |
| R18-S | Spike | R18-CTL | BSA |
| R19-S | Spike | R19-CTL | BSA |
| R20-S | Spike | R20-CTL | BSA |
| R21-S | Spike | R21-CTL | BSA |
| R22-S | Spike | R22-CTL | BSA |
| R23-S | Spike | R23-CTL | BSA |
| R24-S | Spike | R24-CTL | BSA |
| R25-S | Spike | R25-CTL | BSA |

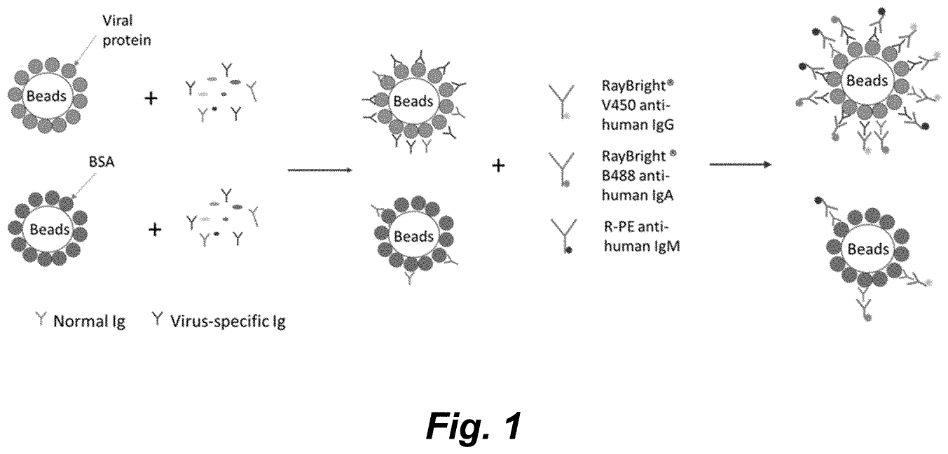

The assay method of the disclosure is a Multiplex Bead Array sandwich-based assay outlined as shown in FIG. 1

One aspect of the disclosure encompasses embodiments of a method of detecting an immune response to SARS-CoV-2, the method comprising: (a) incubating a biological sample from a subject suspected of having been exposed to SARS-CoV-2 virus with a multiplex bead array, wherein the multiplex bead array comprises a plurality of bead populations wherein each bead population is characterized as small, medium, or large based on the size of the beads and grouped according to the intensity of emission of fluorescence therefrom, and wherein the multiplex beads are coated with a protein of SARS-CoV-2, or a fragment thereof, and the sample is incubated individually with a single bead population of the multiplex array of beads; (b) combining and washing the incubated bead populations; (c) adding a mixture of anti-human immunoglobulin (Ig) antibodies to the combined bead populations from step (b), wherein the mixture of anti-human Ig antibodies can comprise at least one anti-human IgG antibody, at least one IgA antibody, and at least one IgM antibody, and wherein the anti-human IgG, IgA, and IgM antibody or antibodies are differentially labeled to distinguish the anti-human IgG antibody or antibodies, the anti-human IgA antibody or antibodies, and the anti-human IgM antibody or antibodies from each other, and wherein the anti-human IgG antibody or antibodies, the anti-human IgA antibody or antibodies, and the anti-human IgM antibody or antibodies can be conjugated to labeling moieties detectable by, and distinguishable from each other, by flow cytometry, and then washing the incubated bead population; (d) repeating steps (a)-(c), wherein the multiplex beads can be coated with a bovine serum albumin, or a fragment thereof; (e) individually analyzing the combined bead populations from steps (c) or (d) with respect to the bead size and the intensity of the emission of a fluorescent signal from each of the different fluorochromes and calculating the ratio of the mean fluorescence intensity (MFI) derived from the results from the combined bead populations coated with a protein of SARS-CoV-2, or a fragment thereof, of step (c) and from the results from the combined bead populations coated with bovine serum albumin of step (d); and (f) determining the relative levels of human IgG, IgA, and IgM bound to the SARS-CoV-2 protein, or a fragment thereof, thereby determining the immune response of the subject to a SARS-CoV-2 infection

In some embodiments of this aspect of the disclosure, the beads can be red fluorescent beads.

In some embodiments of this aspect of the disclosure, each bead population can be in an individual volume in a reaction vessel.

In some embodiments of this aspect of the disclosure, the reaction vessel containing an individual bead population of the multiplex array can be a well of a multiwell-plate.

In some embodiments of this aspect of the disclosure, the method can be a high-throughput assay, wherein each bead population is dispensed into the wells of a single multiwell plate and wherein each well having a bead population coated with a protein of SARS-CoV-2, or a fragment thereof can be paired with a well having a bead population coated with bovine serum albumin.

In some embodiments of this aspect of the disclosure, the protein of SARS-CoV-2 is a SARS-CoV-2 spike protein, a nucleocapsid protein, or a fragment thereof, or any combination thereof.

In some embodiments of this aspect of the disclosure, the protein of SARS-CoV-2 is a SARS-CoV-2 spike protein, or fragment thereof.

In some embodiments of this aspect of the disclosure, the SARS-CoV-2 spike protein is from amino acid positions Arg319 to Phe541 of the receptor-binding domain (RBD)

In some embodiments of this aspect of the disclosure, the labeling moieties can be selected from the group consisting of fluorescein isothiocyanate (FITC, green), Cy2, Cy3, Cy3.5, Cy5, Cy5.5 Cy7, Cy7.5, Texas Red, an Alexa Fluor, a HILYTE™ Fluor, a DYLITE™ Fluor, RayBright® V450, RayBright® B488, and Red Fluorescent Protein (R-PE; R-Phycoerythrin).

In some embodiments of this aspect of the disclosure, the anti-human IgG antibody or antibodies are conjugated to RayBright® V450, the anti-human IgA antibody or antibodies are conjugated to RayBright® B488, and the anti-human IgM antibody or antibodies are conjugated to R-Phycoerythrin.

Another aspect of the disclosure encompasses embodiments of a kit comprising vessels containing a series of size and fluorescent-intensity sorted beads, wherein the beads are coated with at least one polypeptide or fragment thereof derived from SARS-CoV-2, and vessels containing an anti-human IgG antibody, an anti-human IgA antibody, and an anti-human IgM antibody, or antigen-binding fragments thereof, and instructions for the use of the reagents of the kit in a method for the multiplex high-throughput flow cytometry detection of SARS-CoV-2-specific IgG, IgA and IgM antibodies.

In some embodiments of this aspect of the disclosure, the polypeptide or fragment thereof derived from SARS-CoV-2 is at least one of a SARS-CoV-2 spike protein, or a fragment thereof, and a SARS-CoV-2 nucleocapsid protein or a fragment thereof.

In some embodiments of this aspect of the disclosure, the protein of SARS-CoV-2 is a SARS-CoV-2 spike protein.

In some embodiments of this aspect of the disclosure, the SARS-CoV-2 spike protein is from amino acid positions Arg319 to Phe541 of the receptor-binding domain (RBD)

In some embodiments of this aspect of the disclosure, the kit comprises multiplex beads conjugated with SARS-CoV-2 spike protein (R1-S to R25-S), multiplex beads conjugated with bovine serum albumin (R1-CTL to R25-CTL), assay diluent, wash buffer, a V-shaped 96-well microplate, RayBright® V450 labeled goat-anti-human IgG (Fc), RayBright® B488 goat-anti-human IgA (Fc), and R-PE goat-anti-human IgM (Fc).

As mentioned above, compounds of the present disclosure and pharmaceutical compositions can be used in combination of one or more other therapeutic agents for treating viral infection and other diseases. For example, compounds of the present disclosure and pharmaceutical compositions provided herein can be employed in combination with other anti-viral agents to treat viral infection.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

TABLE 2
Assay components

| Description | 3600 Tests | | 18000 Tests | |
|---|---|---|---|---|
| Multiplex Beads Conjugated with | 5.0 | ml/bead | 25.0 | ml/per bead |
| Viral Spike Protein (R1-S to | | | | |
| R25-S) | | | | |
| Multiplex Beads Conjugated with | 5.0 | ml/bead | 25.0 | ml/per bead |
| BSA (R1-CTL to R25-CTL) | | | | |
| 5x Assay Diluent | 50 | ml | 250 | ml |
| 20x Wash Buffer | 50 | ml | 250 | ml |
| V-shaped 96-well Microplate | 25 | | 250 | |
| RayBright.RTM V450 | 600 | μl | 3.0 | ml |
| goat-anti-human | | | | |
| IgG (Fc) | | | | |
| RayBright.RTM B488 | 600 | μl | 3.0 | ml |
| goat-anti-human | | | | |
| IgA (Fc) | | | | |
| R-PE goat-anti-human IgM (Fc) | 600 | μl | 3.0 | ml |

### Additional Requirements

Orbital 96-well plate shaker (with ability to reach 1000 rpm)
Flow Cytometer with Violet, Blue and Red lasers

### Rainbow Calibration Particles

Optional: High Throughput Sampler (HTS) configured for multiple 96-well plate reading

### Example 2

### Assay Protocol

### Preparation of Samples

1 μl of original patient serum, plasma, dilute at 1:8000 in 1× Assay Diluent

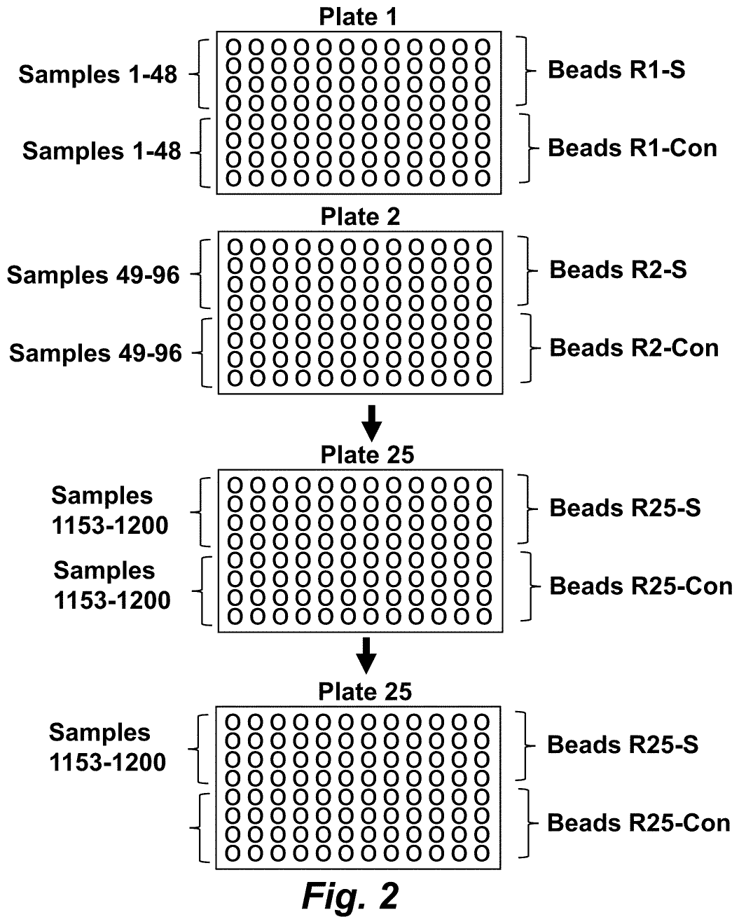

Up to 25 plates (25 set of antigen-bead/control-bead) can be used in one run. A single bead population (based on size of the beads) is used for each of the plates as illustrated in FIG. 2 (total of 25 plates, one for each of R1-R25 beads).

It is preferable to perform a two-step dilution in a 96-well round bottom plate (mirror plate) and then transfer those samples to the test plate. Each test plate (one for each bead size) can test 48 samples, 25 plates (25 set of beads) will be combined in one 96-well plate for testing 1200 samples.

### Preparation of Reagents

All reagents are kept on ice.

The Multiplex beads cocktail must be vortexed for 30 secs each time before use to ensure bead suspension. Use 25 μl beads/test.

Dilute 5× Assay Diluent with deionized H₂O to create an 1× Assay Diluent.

RayBright® V450 goat-anti-human IgG, RayBright® B488 goat-anti-human IgA and R-PE goat-anti-human IgM should be diluted 1:100 in Assay Diluent as working stock. Use 50 μl/test.

Fluorescent multiplex beads should be protected from frequent exposure to light.

Dilute 20× wash buffer with deionized water to create lx wash buffer, diluting only sufficient for the quantity of tests being performed.

### Assay Procedures

1. Prepare up to 25 V-shaped 96-well microplates and mark positions for samples. Each sample is incubated with antigen-beads and control beads in two separate wells.
Advantageously, the top 48 wells (row A-D of a 96-well plate) can be used for antigen bead tests and the lower 48 wells (rows E-H) used for control beads.
2. Add 25 μl of antigen-beads or control-beads to the corresponding wells.
A multi-channel pipet is used to transfer 25 μl pre-diluted samples from the mirror plate to the corresponding wells in the V-shaped test plate. Total volume is each well is 50 μl. The plate is shaken on an orbital shaker at 1000 rpm at room temperature for 90 mins.
3. The beads are washed by adding 200 μl of wash buffer and spun down at 1000 g for 5 minutes at room temperature, followed by removing the supernatant using a multichannel pipet.
4. A multichannel pipet is used to combine the beads of corresponding wells in 25 plates in 200 μl of wash buffer. The combined plate will have 25 beads (R1-S to R25-S, or R1-CTL to R25-CTL) in each well, which are spun down at 1000 g for 5 mins at room temperature. The supernatant is then removed.
6. 50 μl of detection antibodies (RayBright® V450 goat-anti-human IgG, RayBright.RTM B488 goat-anti-human IgA and R-PE goat-anti-human IgM pre-titrated is added to each well and incubated on an orbital shaker at 1000 rpm, room temperature for 30 mins.
7. The plate is washed once as in step 4 and the beads resuspend in 200 μl of Assay Diluent before transfer to a flow cytometer with a high through-put system, or samples are transferred to standard FACS tubes for manually reading.

### Example 3

Flow Cytometry and Data Acquisition: At least two kinds of multiplex beads may be advantageously used in the method of the disclosure, “Red Beads” (emission in the APC channel, or RayBright® 647 channel) and “Blue Beads” (emission in the violet 450nm channel). Red Beads are designated R1-R25.

Three fluorochromes (RayBright® V450, RayBright® B488 and R-PE (Raybiotech Life Inc., Peachtree Corners, Ga., U.S.A.) were used to detect IgM, IgA and IgG simultaneously. Therefore, a flow cytometer with violet, blue and red lasers is required. Standard quality control and optimization for the cytometer are performed during setup.

Compensation is necessary the first time the flow cytometer is used. If beads are too intense in the APC channel, there can be a smiling effect (curved population grouping). When this occurs it is necessary to manually adjust RayBright,RTM V450, RayBright® B488 and R-PE versus APC compensation to correct it. The compensation matrix can be reused if the instrument is standardized with a calibration particle (rainbow beads).

Depending on the model of flow cytometer, it may be necessary to start the acquisition software and run quality control beads before proceeding further.

A new detection run is with RayBright® V450 (equivalent to Pacific Blue or BV421), RayBright® B488 (equivalent to FITC), R-PE and APC (RayBright® 647) channels.

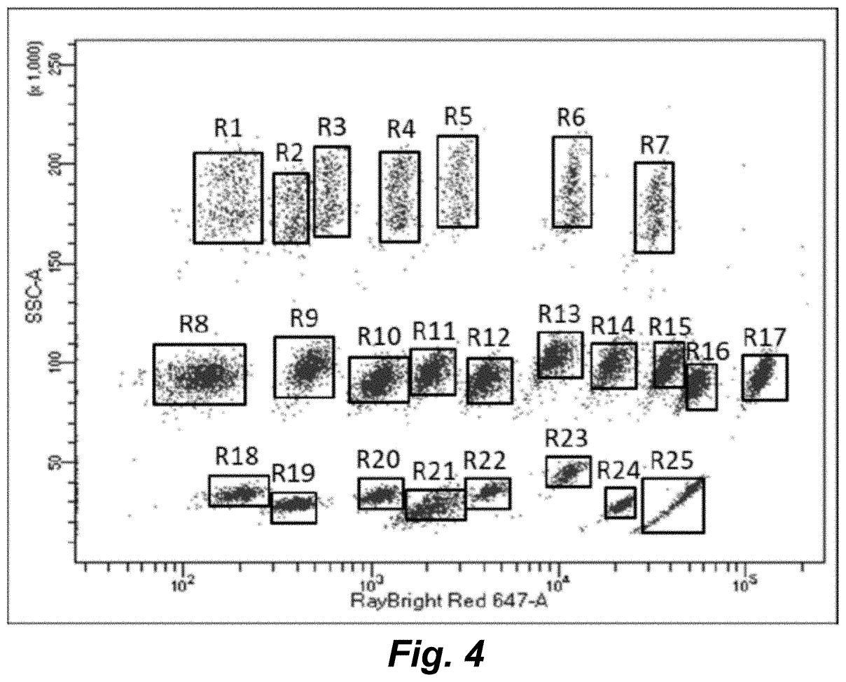

The voltage is adjusted for FSC (forward scatter, linear mode) and SSC (side scatter, linear mode) so that the major bead populations are shown as in FIG. 4. Create FSC-H/FSC-A daughter populations for “Single beads”

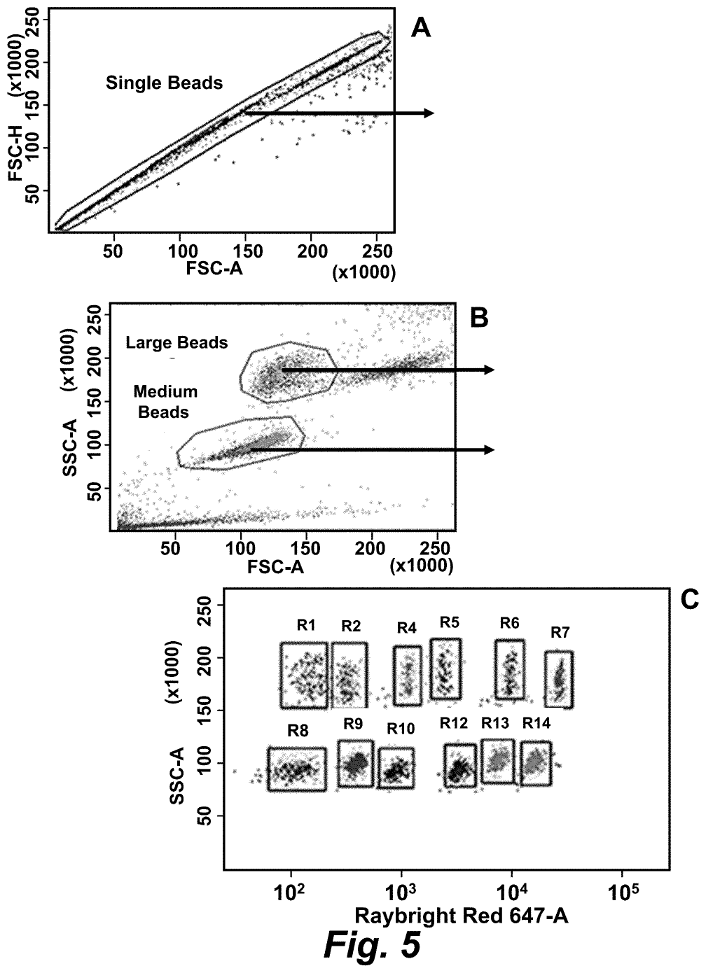

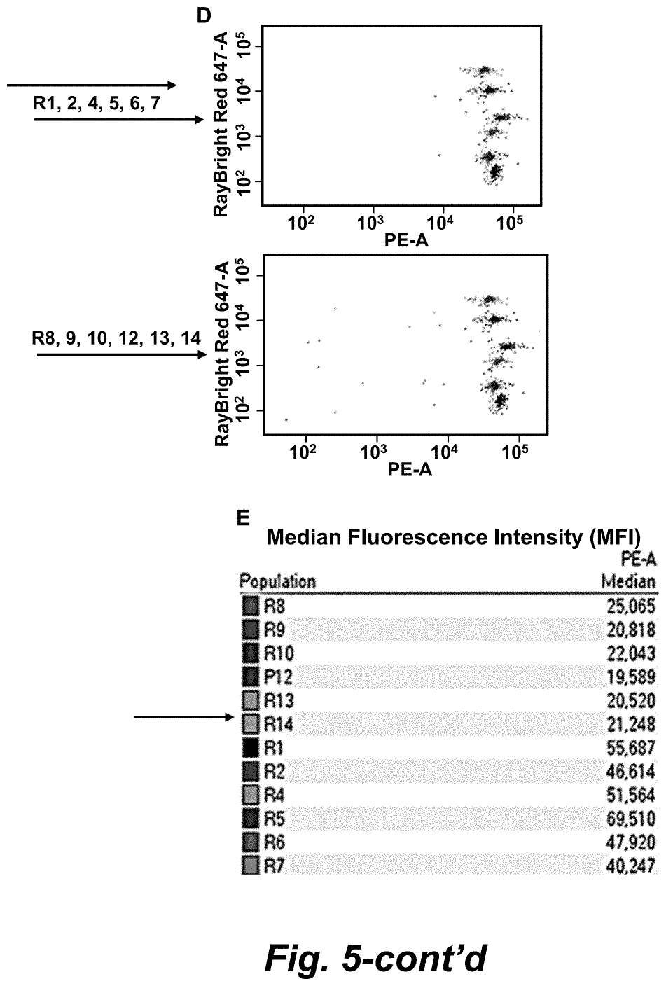

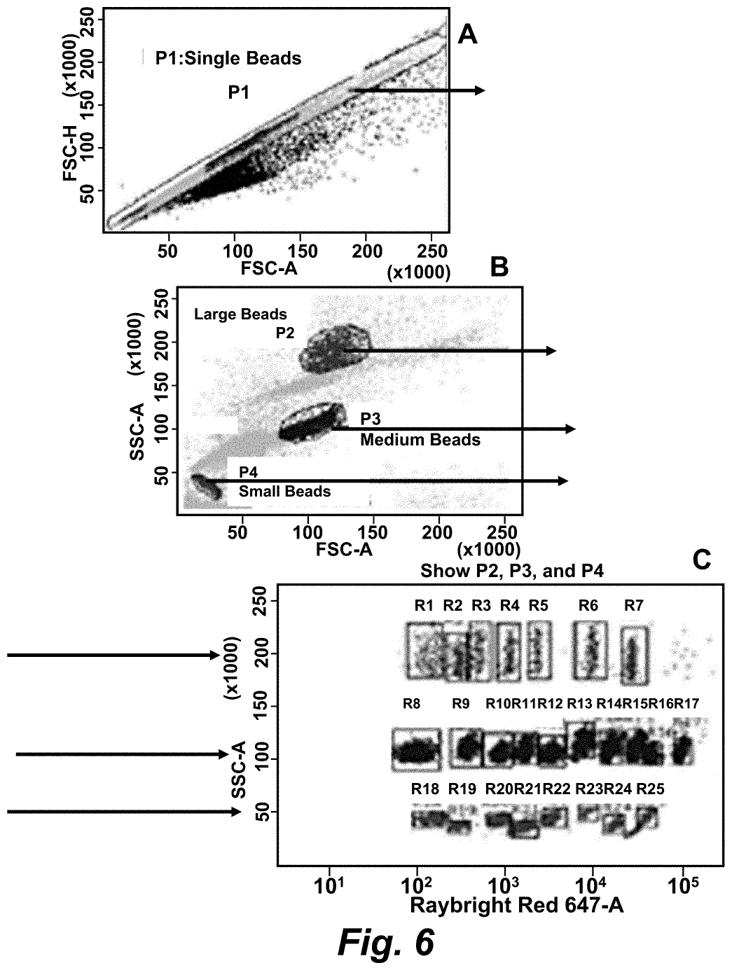

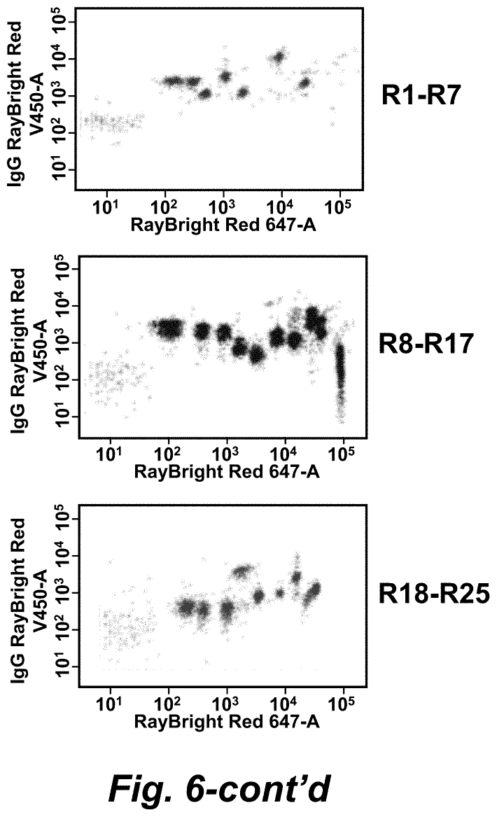

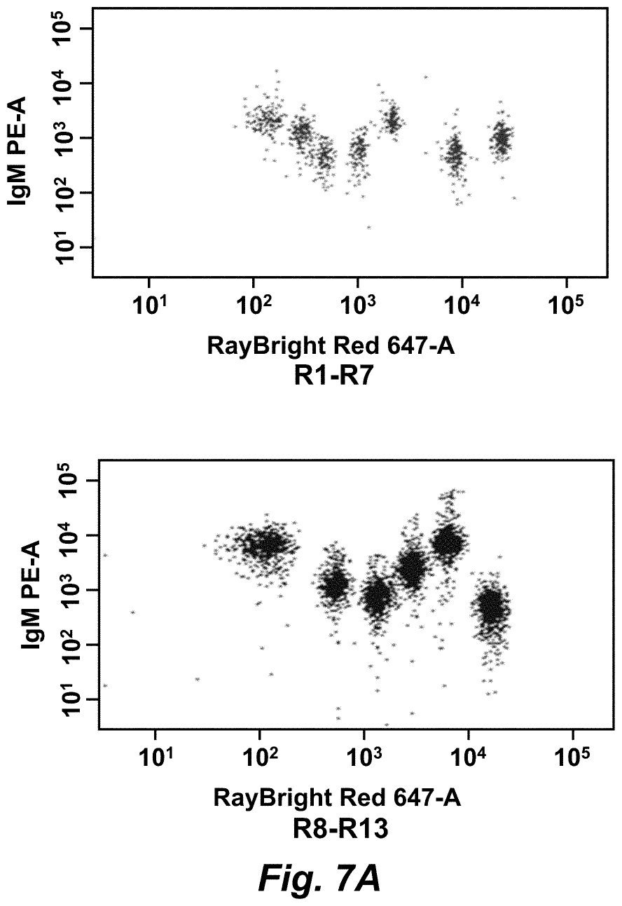

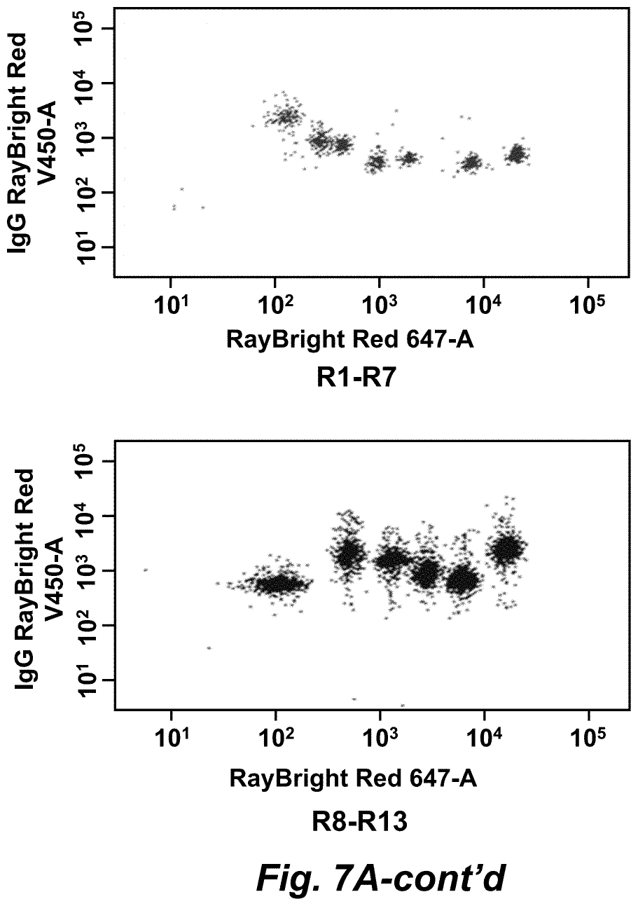

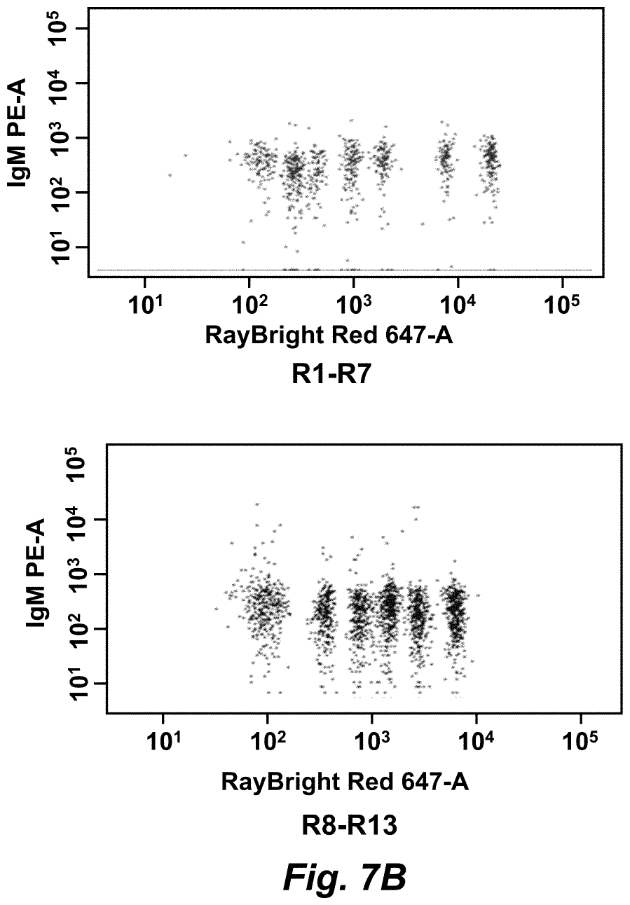

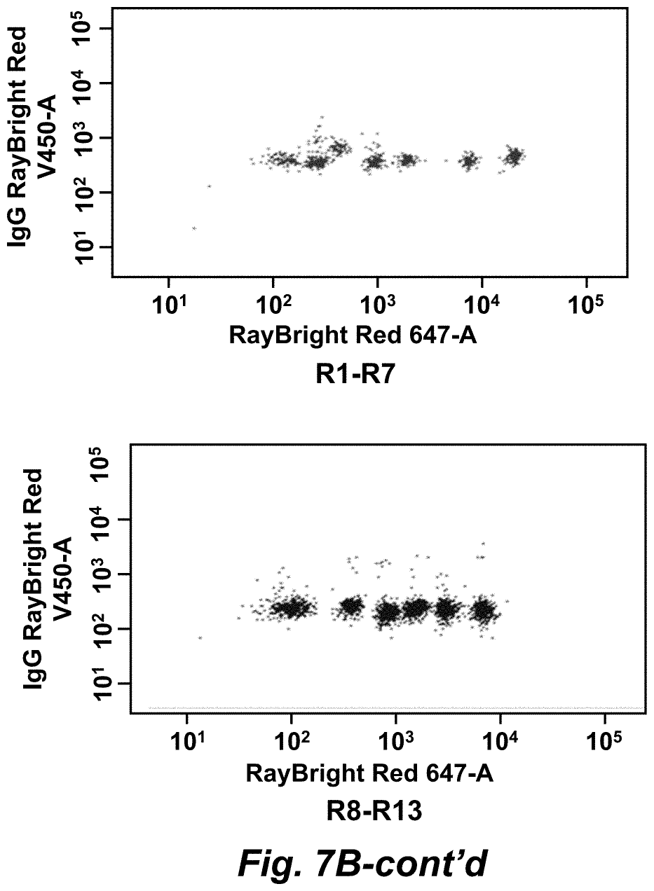

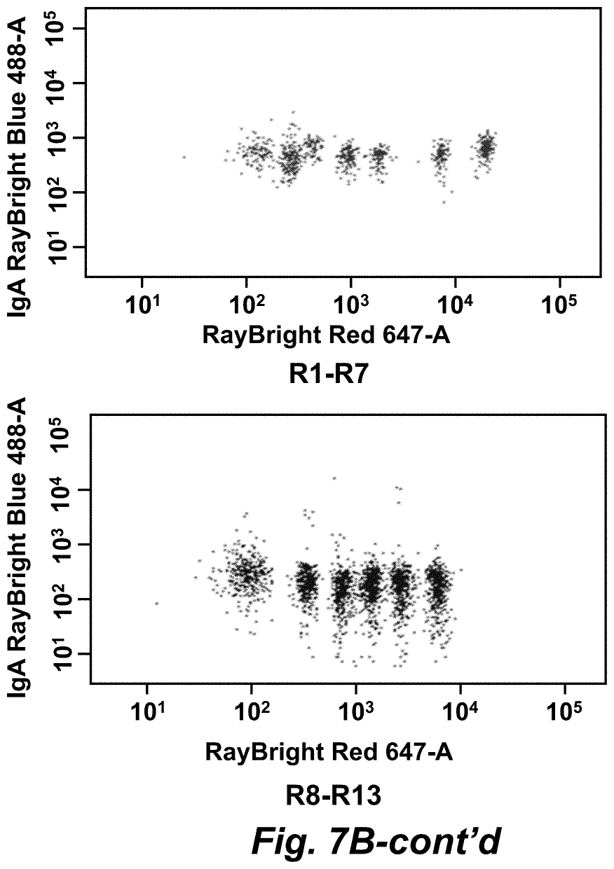

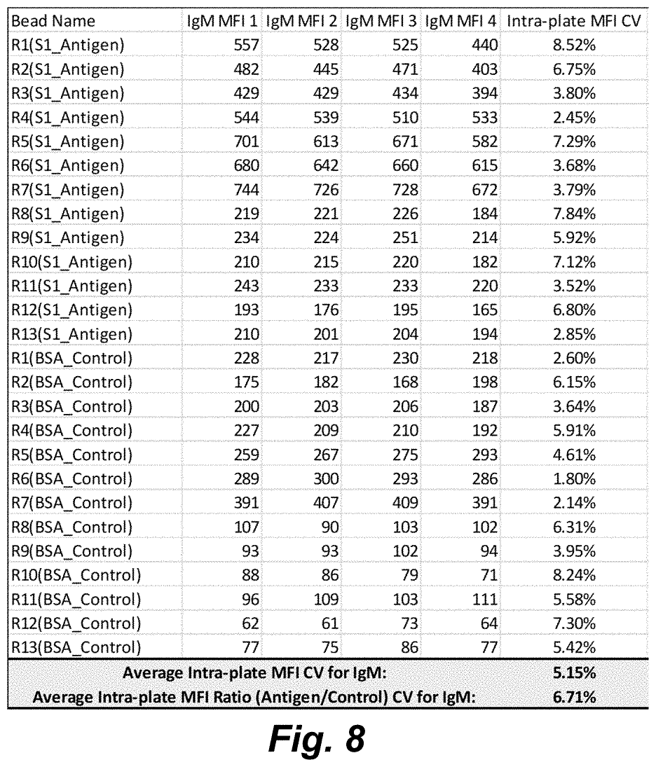

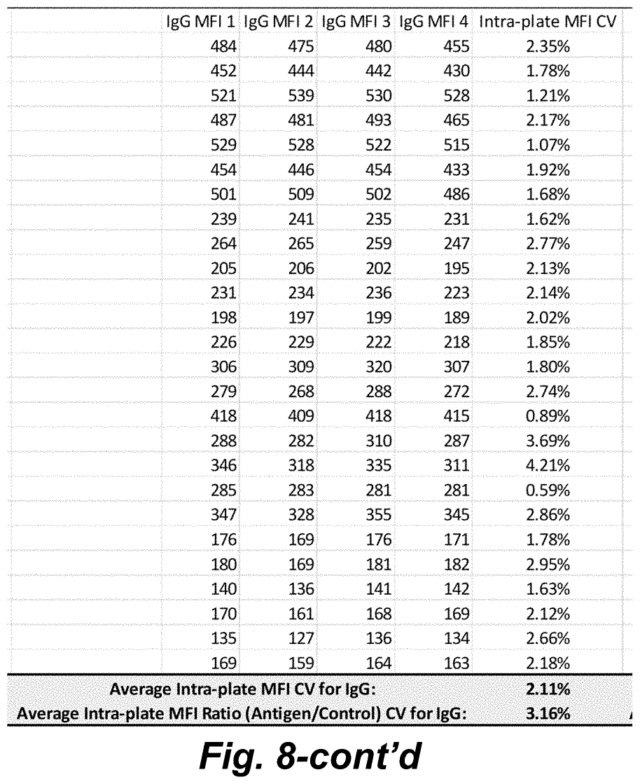

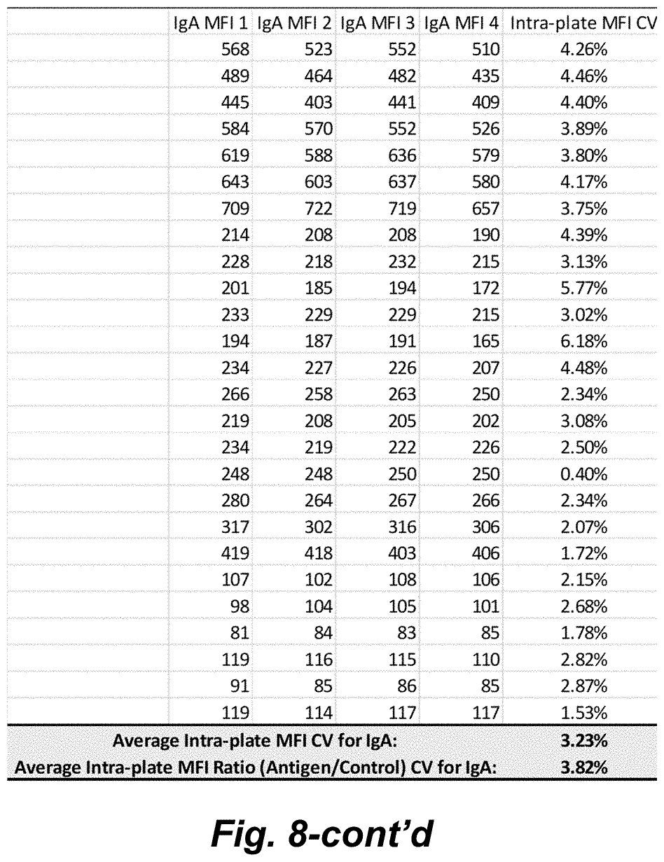

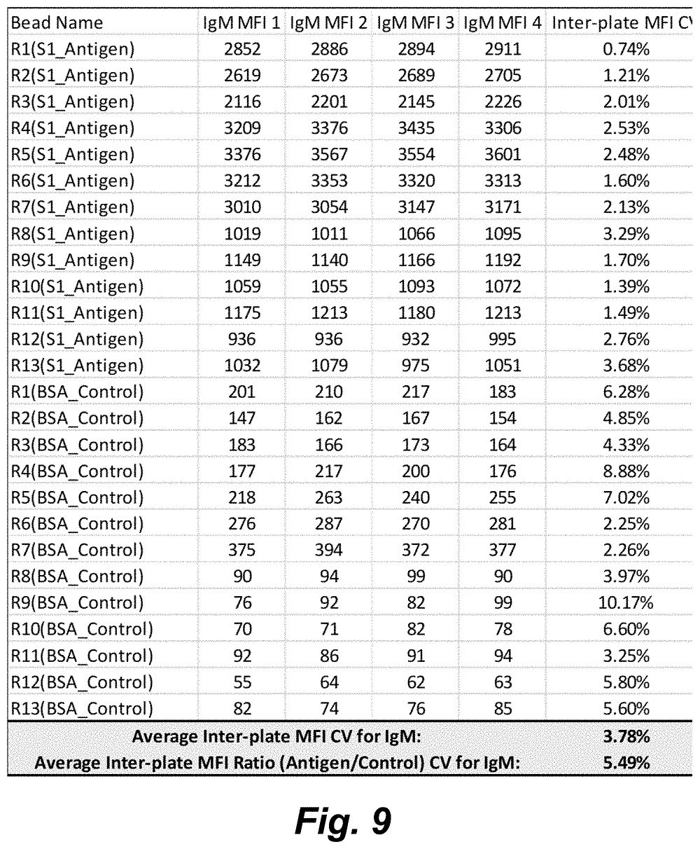

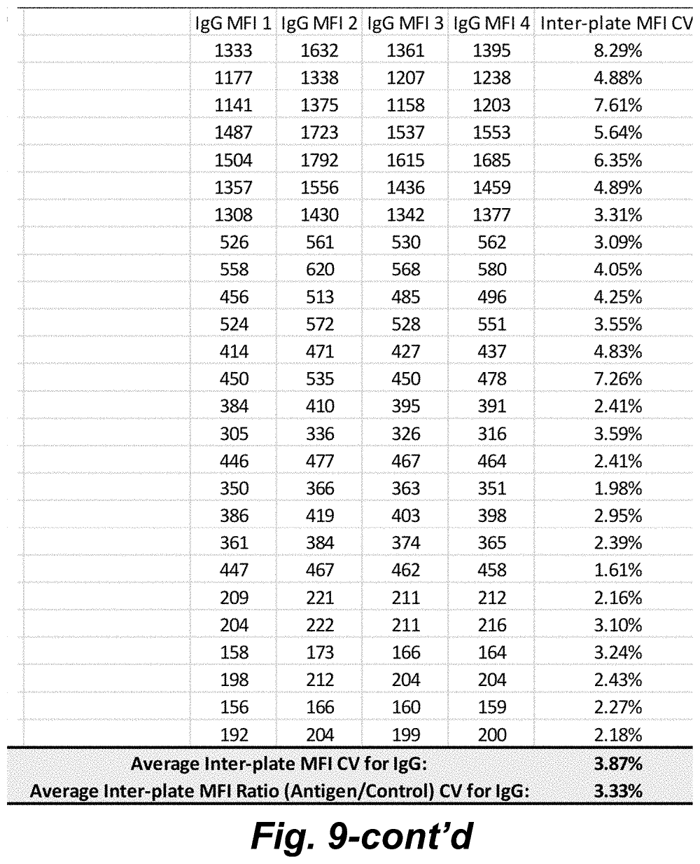

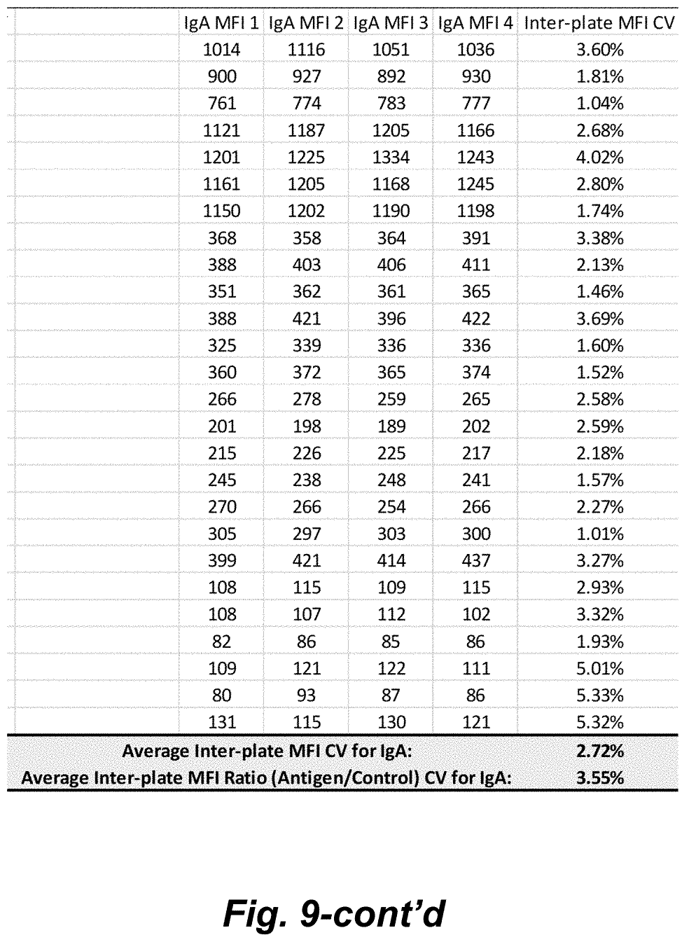

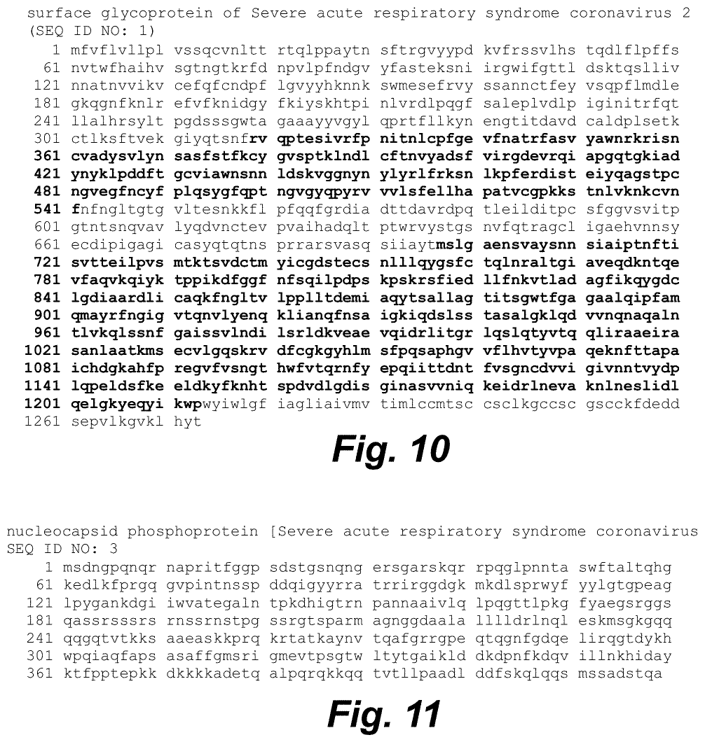

Create a new dot plot from “Single beads” (P1) parent gate, and gate on Large, Medium, and/or Small beads (FIGS. 5 and 6).

Create new dot plot from the Large, Medium, and/or Small beads parent gate. Gate populations for all bead populations (Large beads, P2:R1-R7; Medium beads, P3:R8-R17; and Small beads, P4:R18-R25) for the assay based on SSC (linear mode) and APC (log mode). Voltage is adjusted so that all populations are evenly distributed throughout in a prominent area (FIGS. 5 and 6).

Dot plots Create are using RayBright® V450, RayBright® B488 and R-PE as Y-axis and APC as X-axis (use log scale for both axis) from the large, medium, and small beads parent populations. Run a small amount of the negative beads sample (antigen-beads or control-beads incubated with Assay Diluent only for the first step).

The RayBright® V450, RayBright™ B488 and R-PE voltage is adjusted so that for negative beads for each population the MFI of each channel is about 101-102.

Standard compensation is performed for the 4 colors using compensation beads and any compatible antibody conjugated with the above 4 fluorochromes. It may be necessary to manually adjust compensation of RayBright® V450, RayBright® B488 and R-PE against APC channel to show signals of beads on the far right because the multiplex beads are carrying different intensities of APC fluorescence.

Run samples (as in FIGS. 5 and 6).

Create a statistics view to show MFI for each population (FIGS. 5 and 6).

To keep the testing consistent upon each assay, use of rainbow calibration particles (rainbow beads, mid-range preferred) can allow the standardization of the assay if run in each assay before collecting samples.

After acquiring the data, the entire dataset is exported as an FCS file and export the MFI of RayBright® V450, RayBright® B488 and R-PE for all populations (R1-R25) of all wells with either antigen beads or control beads in Excel format.

### Example 4

Data Analysis using FlowJo: The following bead array data set analysis is based on FlowJo software (BD Life Sciences).

Open FlowJo and drag FCS files or a folder contains FCS files to a new workspace. The example “P1” folder contains a test of 25 antigen bead assay and a test of 25 control bead assay with corresponding 25 serum samples. Save the analysis as a WSP file with a new name.

Choose sample P1_S1(with 25x Antigen Beads coated with SARS-CoV-2 S1 protein), create a “Single beads” gate by FSC-H/FSC-A.

From the “Single beads” population, gate on “Large, Medium and small beads”.

Within the “Large, Medium and small beads” parent gate, create each target bead populations (R1˜R25) using SSC(Linear) by APC(Log). Adjust axis settings to allow separation of targets populations.

For each population, add the “Median” (MFI) statistics for the RayBright® V450, RayBright® B488 and R-PE channel as shown in the picture.

Copy the “Median” (MFI of RayBright® V450, RayBright® B488 and R-PE) population to all target groups (R1-R25).

Copy all gates from P1_BSA to the “All samples” group at the top to apply this gating strategy to all samples.

Click “Table Editor” as shown in the red frame to open the table. Create three tables, Table-IgG, Table-IgA and Table-IgM. For Table-IgG, drag the icon indicating the Median of RayBright® V450 of each population (R1-R25) to the table. Do the same Table-IgA (RayBright® B488) and Table IgM (R-PE)

To export MFI data of IgG for all populations, choose “Group” menu at top of table and pick “P1” group.

In “Output Target” menu, choose “To file”.

In “Output Format” menu, choose “Excel”.

Click “Create Table” icon to export Excel table. Repeat steps 9-13 for Table-IgA and Table-IgM.

### Example 5

Data Interpretation: The antigen beads (R1-S-R25-S) were coated with SARS-CoV-2 antigen S1 and the Control Beads (R1-CTL-R25-CTL) were coated with a control protein (BSA). The conjugation was been optimized so that an antigen bead (e.g. R1-S) and its corresponding control bead (e.g. R1-CTL) had a similar non-specific signal of RayBright® V450, RayBright® B488 and R-PE when incubating with a normal human serum followed by incubation with the above fluorochrome-conjugated detection antibody against human IgG, IgA and IgM.

The ratio of the MFI of RayBright® V450, RayBright® B488 and R-PE of antigen bead versus control bead were used to determine whether the serum sample was positive for anti-viral IgG, IgA or IgM. As indicated, any ratio of 2 and above was diagnosed as positive (shown in red numbers), while any ratio of 1 or less was diagnosed as negative.

### Example 6

Table 3 shows the determination of positivity based on MFI ration between Antigen Beads and Control Beads. Exported MFI for RayBright® V450 anti-human IgG, RayBright® B488 anti-human IgA and R-PE anti-human IgM of each sample from antigen beads and control beads.

TABLE 3

| | | | | | | | | | | | | | |
|---|---|---|---|---|---|---|---|---|---|---|---|---|---|
| Bead ID | R1 | R2 | R3 | R4 | R5 | R6 | R7 | R8 | R9 | R10 | R11 | R12 | R13 |

MFI of R-PE (IgM)

| | | | | | | | | | | | | | |
|---|---|---|---|---|---|---|---|---|---|---|---|---|---|
| P1_S1(Antigen Beads) | 2327 | 1337 | 472 | 615 | 2391 | 521 | 1067 | 8569 | 1962 | 1141 | 3575 | 9828 | 703 |
| P1_BSA(Control Beads) | 342 | 197 | 197 | 299 | 331 | 396 | 363 | 169 | 101 | 79.9 | 132 | 69.2 | 91 |

MFI of RayBright.RTM V450 (IgG)

| | | | | | | | | | | | | | |
|---|---|---|---|---|---|---|---|---|---|---|---|---|---|
| P1_S1(Antigen Beads) | 3283 | 1194 | 995 | 488 | 563 | 473 | 684 | 609 | 2176 | 1571 | 1004 | 703 | 2465 |
| P1_BSA(Control Beads) | 429 | 390 | 694 | 400 | 416 | 415 | 515 | 226 | 238 | 182 | 222 | 208 | 207 |

MFI of RayBright ® B488 (IgA)

| | | | | | | | | | | | | | |
|---|---|---|---|---|---|---|---|---|---|---|---|---|---|
| P1_S1(Antigen Beads) | 1552 | 1047 | 602 | 430 | 563 | 556 | 758 | 705 | 4075 | 971 | 529 | 6475 | 709 |
| P1_BSA(Control Beads) | 404 | 292 | 529 | 338 | 350 | 379 | 485 | 251 | 163 | 112 | 157 | 141 | 141 |

Table 4 shows the ratio of MFI for RayBright® V450 anti-human IgG, RayBright.RTM B488 anti-human IgA and R-PE anti-human IgM of each sample between Antigen Beads and Control Beads.

TABLE 4

| Bead ID | | | | | | | | | | | | |
|---|---|---|---|---|---|---|---|---|---|---|---|---|
| R1 | R2 | R3 | R4 | R5 | R6 | R7 | R8 | R9 | R10 | R11 | R12 | R13 |
| Sample ID | | | | | | | | | | | | |
| S0001 | S0002 | S0003 | S0004 | S0005 | S0006 | S0007 | S0008 | S0009 | S0010 | S0011 | S0012 | S0013 |

Ratio of MFI of R-PE (IgM)

| | | | | | | | | | | | | | |
|---|---|---|---|---|---|---|---|---|---|---|---|---|---|
| P1_S1(Antigen Beads) | 2327 | 1337 | 472 | 615 | 2391 | 521 | 1067 | 8569 | 1962 | 1141 | 3575 | 9828 | 703 |
| P1_BSA(Control Beads) | 342 | 197 | 197 | 299 | 331 | 396 | 363 | 169 | 101 | 79.9 | 132 | 69.2 | 91 |
| Ratio | 6.8 | 6.8 | 2.4 | 2.1 | 7.2 | 1.3 | 2.9 | 50.7 | 19.4 | 14.3 | 27.1 | 142.0 | 7.7 |

Ratio ofMFI of RayBright ® V450 (IgG)

| | | | | | | | | | | | | | |
|---|---|---|---|---|---|---|---|---|---|---|---|---|---|
| P1_S1(Antigen Beads) | 3283 | 1194 | 995 | 488 | 563 | 473 | 684 | 609 | 2176 | 1571 | 1004 | 703 | 2465 |
| P1_BSA(Control Beads) | 429 | 390 | 694 | 400 | 416 | 415 | 515 | 226 | 238 | 182 | 222 | 208 | 207 |
| Ratio | 7.7 | 3.1 | 1.4 | 1.2 | 1.4 | 1.1 | 1.3 | 2.7 | 9.1 | 8.6 | 4.5 | 3.4 | 11.9 |

Ratio ofMFI of RayBright ® B488 (IgA)

| | | | | | | | | | | | | | |
|---|---|---|---|---|---|---|---|---|---|---|---|---|---|
| P1_S1(Antigen Beads) | 1552 | 1047 | 602 | 430 | 563 | 556 | 758 | 705 | 4075 | 971 | 529 | 6475 | 709 |
| P1_BSA(Control Beads) | 404 | 292 | 529 | 338 | 350 | 379 | 485 | 251 | 163 | 112 | 157 | 141 | 141 |
| Ratio | 3.8 | 3.6 | 1.1 | 1.3 | 1.6 | 1.5 | 1.6 | 2.8 | 25.0 | 8.7 | 3.4 | 45.9 | 5.0 |

Table 5 shows the sensitivity and specificity of MFI ratio-based assay. A. Cutoff value for positive sample are determined by MFI ratio equal or over than 1.5 and S1 antigen bead MFI reading over 200. B. Sensitivity is calculated as True positive/(True Positive+False Negative); Specificity is calculated as True Negative/(True Negative+False Positive).

TABLE 5

| | | | |
|---|---|---|---|
| Ratio Cutoff Used: | 1.5 | MFI Cutoff Used | 200 |
| Total Patient Samples: | 121 | Total Normal Samples: | 299 |
| False Negative: | 2 | False Positive: | 2 |
| Sensitivity: | 97.5% | Specificity: | 99.3% |

### Example 6

Shown are the SARS-CoV-2 antigens that may be used in the methods of the disclosure.

TABLE 6
Recombinant Spike Subunit 1 (S1) Protein

| Protein Domain | Expression Host | Expression Region |
|---|---|---|
| Receptor-Binding | HEK293 Cell | Arg319 - Phe541 (SEQ ID NO: 2) |
| Domain | HEK293 Cell | Arg319 - Phe541 (N331Q mutant) |
| (RBD) | E. coli | Arg319 - Phe541 |
| Full length | E. coli | Val16 - Gln690 from SEQ ID |
| | | NO: 1) |

### Recombinant Spike Subunit 2 (S2) Protein

| Protein Domain | Expression Host | Expression Region |
|---|---|---|
| Full length | E. coli | Met697 - Pro1213 (SEQ ID NO: 3) |
| | HEK293 Cell | Met697 - Pro1213 |

What is claimed:

1. A method of detecting an immune response to SARS-CoV-2, the method comprising:
(a) incubating a biological sample from a subject suspected of having been exposed to SARS-CoV-2 virus with a multiplex bead array, wherein the multiplex bead array comprises a plurality of bead populations wherein each bead population is characterized as small, medium, or large based on the size of the beads and grouped according to the intensity of emission of fluorescence therefrom, and wherein the multiplex beads are coated with a protein of SARS-CoV-2, or a fragment thereof, and the sample is incubated individually with a single bead population of the multiplex array of beads;
(b) combining and washing the incubated bead populations;
(c) adding a mixture of anti-human immunoglobulin (Ig) antibodies to the combined bead populations from step (b), wherein the mixture of anti-human Ig antibodies comprises at least one anti-human IgG antibody, at least one IgA antibody, and at least one IgM antibody, and wherein the anti-human IgG, IgA, and IgM antibody or antibodies are differentially labeled to distinguish the anti-human IgG antibody or antibodies, the anti-human IgA antibody or antibodies, and the anti-human IgM antibody or antibodies from each other, and wherein the anti-human IgG antibody or antibodies, the anti-human IgA antibody or antibodies, and the anti-human IgM antibody or antibodies are conjugated to different fluorochromes detectable by, and distinguishable from each other, by flow cytometry, and then washing the incubated bead population;
(d) repeating steps (a)-(c), wherein the multiplex beads can be coated with a bovine serum albumin, or a fragment thereof;
(e) individually analyzing the combined bead populations from steps (c) or (d) with respect to the bead size and the intensity of the emission of a fluorescent signal from each of the different fluorochromes and calculating the ratio of the mean fluorescence intensity (MFI) derived from the results from the combined bead populations coated with a protein of SARS-CoV-2, or a fragment thereof, of step (c) and from the results from the combined bead populations coated with bovine serum albumin of step (d); and
(f) determining the relative levels of human IgG, IgA, and IgM bound to the SARS-CoV-2 protein, or a fragment thereof, thereby determining the immune response of the subject to a SARS-CoV-2 infection.

2. The method of claim 2, wherein the beads are red fluorescent beads.

3. The method of claim 1, wherein each bead population is in an individual volume in a reaction vessel.

4. The method of claim 3, wherein the reaction vessel containing an individual bead population of the multiplex array is a well of a multiwell-plate.

5. The method of claim 1, wherein the method is a high-throughput assay, wherein each bead population is dispensed into the wells of a single multiwell plate and wherein each well having a bead population coated with a protein of SARS-CoV-2, or a fragment thereof is paired with a well having a bead population coated with bovine serum albumin.

6. The method of claim 1, wherein the protein of SARS-CoV-2 is a SARS-CoV-2 spike protein, a nucleocapsid protein, or a fragment thereof, or any combination thereof.

7. The method of claim 1, wherein the protein of SARS-CoV-2 is a SARS-CoV-2 spike protein, or a fragment thereof.

8. The method of claim 7, wherein the SARS-CoV-2 spike protein is from amino acid positions Arg319 to Phe541 (SEQ ID NO: 2) of the receptor-binding domain (RBD).

9. The method of claim 1, wherein the labeling moieties are selected from the group consisting of fluorescein isothiocyanate (FITC, green), Cy2, Cy3, Cy3.5, Cy5, Cy5.5 Cy7, Cy7.5, Texas Red, an Alexa Fluor, a HILYTE™ Fluor, a DYLITE™ Fluor, RayBright® V450, RayBright® B488, and Red Fluorescent Protein (R-PE; R-Phycoerythrin).

10. The method of claim 1, wherein the anti-human IgG antibody or antibodies are conjugated to RayBright® V450, the anti-human IgA antibody or antibodies are conjugated to RayBright® B488, and the anti-human IgM antibody or antibodies are conjugated to R-Phycoerythrin.

11. A kit comprising vessels containing a series of size and fluorescent-intensity sorted beads, wherein the beads are coated with at least one polypeptide or fragment thereof derived from SARS-CoV-2, and vessels containing an anti-human IgG antibody, an anti-human IgA antibody, and an anti-human IgM antibody, or antigen-binding fragments thereof, and instructions for the use of the reagents of the kit in the method of claim 1 for the multiplex high-throughput flow cytometry detection of SARS-CoV-2-specific IgG, IgA and IgM antibodies.

12. The kit of claim 11, wherein the polypeptide or fragment thereof derived from SARS-CoV-2 is at least one of a SARS-CoV-2 spike protein, or a fragment thereof, and a SARS-CoV-2 nucleocapsid protein or a fragment thereof. 13, The kit of claim 12, wherein the kit comprises multiplex beads conjugated with SARS-CoV-2 spike protein (R1-S to R25-S), multiplex beads conjugated with bovine serum albumin (R1-CTL to R25-CTL), assay diluent, wash buffer, a V-shaped 96-well microplate, RayBright® V450 labeled goat-anti-human IgG (Fc), RayBright® B488 goat-anti-human IgA (Fc), and R-PE goat-anti-human IgM (Fc).

14. The kit of claim 11, wherein the protein of SARS-CoV-2 is a SARS-CoV-2 spike protein.

15. The kit of claim 11, wherein the SARS-CoV-2 spike protein is from amino acid positions Arg319 to Phe541 of the receptor-binding domain (RBD).


SEQUENCE LISTING (4 sequences)

SEQ ID NO: 1 - protein, 1273 aa, Artificial sequence
  surface spike glycoprotein of SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 223 aa, Artificial sequence
  amino acids R319-F541 of the receptor binding domain (RBD) of surface spike glycoprotein of SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 3 - protein, 518 aa, Artificial sequence
  Amino acids M697-P1213 of the S2 region of the surface spike glycoprotein of SARS-CoV-2
MSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPF

SEQ ID NO: 4 - protein, 419 aa, Artificial sequence
  Amino acids M1-A419 of the SARS-CoV-2 nucleocapsid protein.
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA